(19) United States
(12) Patent Application Publication
(10) Pub. No.: US 2025/0352132 A1
(43) Pub. Date: Nov. 20, 2025

(54) METHOD, APPARATUS AND COMPUTER PROGRAM PRODUCT FOR GENERATING A QUANTITATIVE NEUROMUSCULAR BLOCKADE ASSESSMENT USING COMPUTER VISION

(71) Applicant: University of Florida Research Foundation, Incorporated, Gainesville, FL (US)

(72) Inventors: Adam Wolach, Gainesville, FL (US); Nikolaus Gravenstein, Gainesville, FL (US)

(21) Appl. No.: 19/181,717

(22) Filed: Apr. 17, 2025

Related U.S. Application Data
(60) Provisional application No. 63/635,142, filed on Apr. 17, 2024.

(51) Int. Cl. A61B 5/00; A61B 5/11

(52) U.S. Cl. CPC A61B 5/4519; A61B 5/0077; A61B 5/1107; A61B 5/4041; A61B 5/742; A61B 5/746

(57) ABSTRACT

A method, apparatus, and computer program product are provided for generating quantitative neuromuscular blockade assessments. Image data captured from a camera, such as a camera integrated in a mobile device is applied to a computer vision model to generate body part movement data indicating positions of the body parts in a three-dimensional space. Using the movement data, a twitch count or train of four (TOF) ratio is calculated and used to generate a quantitative neuromuscular blockade assessment. A notification is generated and provided via a user interface based on the neuromuscular blockade assessment. The quantitative neuromuscular blockade assessment can indicate a depth of neuromuscular blockade, maintenance of neuromuscular blockade, adequate recovery from neuromuscular blockade, presence of residual blockade, a calculated dosage of a maintenance or reversal agent, or the like.

### CROSS REFERENCES TO RELATED APPLICATION

This application claims the benefit of priority to U.S. Provisional Application No. 63/635,142, filed Apr. 17, 2024, the entire contents of which are hereby incorporated by reference.

### TECHNOLOGICAL FIELD

An example embodiment of the present invention relates generally to computer vision, and more particularly, to a method, apparatus and computer program product for generating a quantitative depth of neuromuscular blockade determination.

### BACKGROUND

Neuromuscular blockade is frequently used in anesthesia, for surgery, intubation, and other medical procedures. Residual neuromuscular blockade after a procedure increases the risk of aspiration events and reintubation. It is vital for a patient's health to eliminate residual blockade with either adequate time for spontaneous recovery or through chemical reversal. Additionally, it is essential to ensure a patient's recovery from neuromuscular blockade prior to extubation, as a patient may be too weak or unable to breathe adequately, when residual neuromuscular blockade is present. For example, when a patient is recovering from surgery, and extubated too early, in the presence of residual neuromuscular blockade the patient's tongue may impede proper breathing, there may be inadequate airway protection, there is an increased requirement for supplemental oxygen administration, etc.

The train of four (TOF) ratio is an indicator of neuromuscular blockade depth and involves the delivery of four electrical stimulations at 2 hz to a target nerve, typically the ulnar nerve at the wrist, to produce detectable muscle twitches. The ratio of measured excursion of the fourth to the first twitch is correlated with the percentage of blocked acetylcholine receptors, providing a non-invasive measure of the depth of, or degree of recovery from, neuromuscular blockade.

To monitor the TOF ratio, a clinician initiates a transcutaneous electrical stimulus to a motor nerve via a peripheral nerve stimulator. The stimulation causes the innervated muscles to twitch, and the clinician observes and/or feels the ratio in amplitude of the fourth twitch compared to the first of a series of four twitches. The ratio is used to assess neuromuscular blockade, and drives decisions relating to establishment, maintenance, and achievement of acceptable recovery from neuromuscular blockade. For example, a TOF ratio of 90% or greater relative to baseline is needed for safe extubation and recovery after surgery. In contrast a twitch count of 1 or 2 or even a TOF ratio of 15-25% may indicate adequate surgical relaxation depending on the procedure.

Given the cruciality of the TOF ratio, quantitative measuring techniques are desired. Some implementations have been developed to provide a quantitative measurement technique in comparison to earlier and more prevalent observation-based methods. Electromyography (EMG) uses electrodes to detect the electrical activity of the muscle in response to stimulation. Kinemyography (KMG) uses a piezoelectric sensor to measure voltage in the sensor. Phonomyography (PMG) uses a high-fidelity narrow-bandwidth microphone placed along the muscle to measure sound intensity. Acceleromyography (AMG) uses piezoelectric sensors to measure acceleration of the muscles. Such methods offer improved accuracy over observation-based methods but require extensive overhead with regard to infrastructure and equipment. Some sensors are expensive, and may include single-use or disposable electrodes, further increasing operational costs. Some sensor types require regular calibration and maintenance of electronic components. The need for dedicated monitoring devices further increases the cost, and clinicians need special training to properly place the sensors to obtain accurate readings.

### BRIEF SUMMARY

A method, apparatus, and computer program product are therefore provided for generating a quantitative neuromuscular blockade assessment using a computer vision model. Example embodiments provided herein utilize computer vision to calculate the TOF ratio and generate quantitative neuromuscular blockade assessments accordingly. Example embodiments reduce error in such measurements taken by conventional clinical methods, such as a clinician visually and or tactilely estimating, a TOF ratio. In this regard, example embodiments use machine learning and computer vision algorithms to track the movement of certain body parts, such as the thumb and fifth finger, on a patient under medical observation. Example embodiments may use a library of hand images for training the system to recognize the first and fifth fingers from a view of the hand. Example embodiments then track movements thereof to calculate the ratio of magnitude between the first and fourth twitches or excursions (the TOF ratio).

Using machine learning algorithms enables example embodiments to account for varying skin color, lighting conditions, anatomy, or the like. Example embodiments can be implemented on a mobile device or other device with a camera and may be expanded to measure movements in the ankle, feet and eye that are responsive to nerve stimulation. Example embodiment can optionally use a fiducial marker to track movement of body parts.

A system is provided, including memory and one or more processors communicatively coupled to the memory, the one or more processors configured to receive image data captured from one or more cameras, and apply a computer vision model to generate body part movement data, wherein the body part movement data indicate positioning of one or more body parts in a three-dimensional space. The one or more processors are further configured to calculate a train of four (TOF) ratio using the body part movement data, and, based upon the calculated TOF ratio, generate a quantitative i.e numerical neuromuscular blockade assessment.

According to certain example embodiments, the one or more processors are further configured to generate and provide a notification based on the neuromuscular blockade assessment. According to certain example embodiments, the one or more processors are further configured to train the computer vision model using one or more simulated body parts and generated video frames of the one or more simulated body parts. The one or more processors may be further configured to receive and monitor additional image data, wherein the neuromuscular blockade assessment is generated based on a baseline TOF ratio and the additional image data. The neuromuscular blockade assessment comprises a quantitative assessment, and may include a calculated predicted recommended dosage of reversal agent. The one or more processors are further configured to cause display of the calculated dosage of the reversal agent via a user interface.

A computer-implemented method is provided, including, receiving image data captured from one or more cameras, and applying a computer vision model to generate body part movement data, wherein the body part movement data indicate positioning of one or more body parts in a three-dimensional space. The method further includes calculating a train of four (TOF) ratio using the body part movement data, and based upon the calculated TOF ratio, generating a quantitative neuromuscular blockade assessment. The method further includes generating and providing a notification based on the neuromuscular blockade assessment.

According to certain embodiments, the method includes training the computer vision model using one or more simulated body parts and generated video frames of the one or more simulated body parts. The method may further include receiving and monitoring additional image data, wherein the neuromuscular blockade assessment is generated based on a baseline TOF ratio and the additional image data. The computer-implemented method further includes causing display of the calculated dosage of reversal agent via a user interface.

A computer program product is provided, comprising at least one non-transitory computer-readable storage medium having computer-executable program code instructions stored therein, the computer-executable program code instructions comprising program code instructions to receive image data captured from one or more cameras, apply a computer vision model to generate body part movement data, wherein the body part movement data indicate positioning of one or more body parts in a three-dimensional space, calculate a train of four (TOF) ratio using the body part movement data, and based upon the calculated TOF ratio, generate a quantitative neuromuscular blockade assessment. The computer-executable program code instructions further comprise program code instructions to generate and provide a notification based on the neuromuscular blockade assessment.

According to certain embodiments, the computer-executable program code instructions further comprise program code instructions to train the computer vision model using one or more simulated body parts and generated video frames of the one or more simulated body parts.

According to certain embodiments, the computer-executable program code instructions further comprise program code instructions to receive and monitor additional image data, wherein the neuromuscular blockade assessment is generated based on a baseline TOF ratio, also referred to as a normalized TOF ratio, and the additional image data.

### BRIEF DESCRIPTION OF THE DRAWINGS

Having thus described certain example embodiments of the present invention in general terms, reference will hereinafter be made to the accompanying drawings which are not necessarily drawn to scale, and wherein:

FIG. 1 is an overview of a system that can be used to practice certain example embodiments disclosed herein;

FIG. 2 is an example schematic diagram of an apparatus according to certain example embodiments disclosed herein;

FIG. 3 illustrates an example mobile device displaying a TOF ratio of 95% in accordance with certain example embodiments disclosed herein;

FIG. 4 is an example schematic of a body part recognized and tracked in accordance with certain example embodiments disclosed herein;

FIGS. 5-6 are flowcharts illustrating operations performed in accordance with some example embodiments;

FIG. 7 is an example simulated environment in accordance with certain example embodiments disclosed herein; and

FIG. 8 is a plot of data in accordance with certain example embodiments disclosed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout. Moreover, while certain embodiments of the present disclosure are described with reference to predictive data analysis, one of ordinary skill in the art will recognize that the disclosed concepts can be used to perform other types of data analysis.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

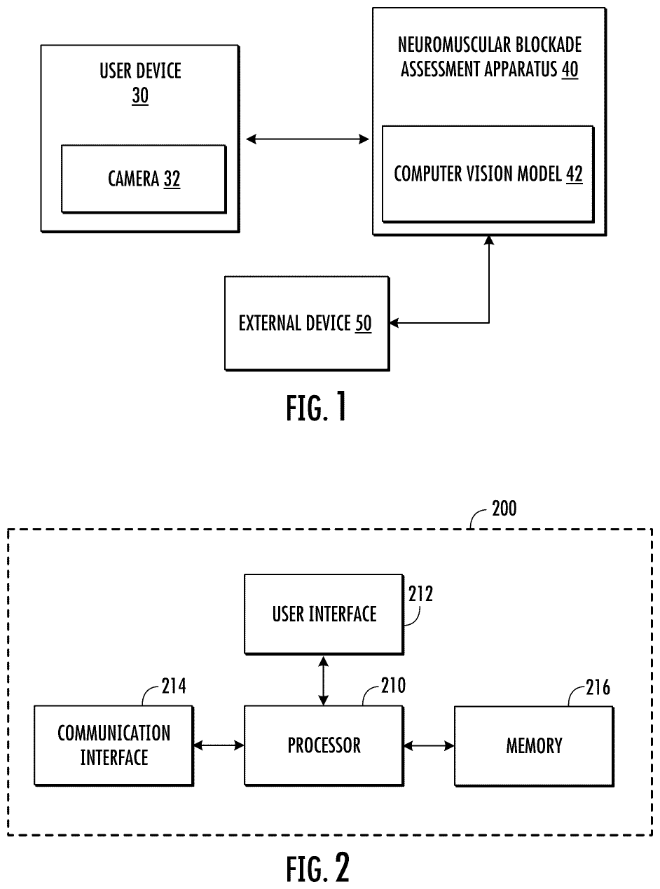

As described below, a method, apparatus and computer program product are provided for generating a quantitative neuromuscular blockade assessment using a computer vision model. FIG. 1 is an overview of a system that can be used to practice certain embodiments described herein and should not be considered limiting.

As illustrated in FIG. 1, example embodiments may be implemented as or employed in a distributed system. The various depicted components may be configured to communicate over a network, such as the Internet, for example, or any other communication interface as described in further detail hereinafter. User device 30 may include a smart phone, tablet, notebook, laptop computer, or any other suitable computing device. The user device 30 may include or may be communicatively connected to one or more cameras 32. The user device 30 can be controlled by a user, such as a nurse, clinician, or other medical practitioner, to enable the camera 32 to capture image data, such as images or video of a patient's hand or other body part. The image data are transmitted to the quantitative neuromuscular blockade assessment apparatus 40 via the user device 30.

Additionally, or alternatively, a camera 32 that is peripheral from the user device 30 may be positioned in a room to capture the image data. The user device 30 may be used to provide, via a user interface, an indication of the neuromuscular blockade assessment, such as may be communicated by the neuromuscular blockade assessment apparatus 40. For example, the user device 30 may indicate a TOF ratio, an alert regarding the neuromuscular blockade assessment, or the like.

The neuromuscular blockade assessment apparatus 40 is a predictive data analysis computing entity and can include a server, computer workstation, personal computer, smart phone, or any suitable computing device, and is configured to receive image data and provide a quantitative neuromuscular blockade assessment accordingly. The neuromuscular blockade assessment apparatus 40 includes a computer vison model 42, trained to recognize and track moving body parts, and calculate the TOF ratio. The computer vision model 42 may be trained with image frames of body parts, such as the human hand, using one or more convolutional neural networks (CNNs) to provide real time accurate three-dimensional hand, or other appropriate body part, motion tracking.

For example, OpenCV, an opensource CNN, may be utilized to perform certain operations relating to frame ingestion, processing, labeling, and display. GOOGLE MEDIAPIPE may be utilized to provide cross-platform hand tracking, including single shot palm recognition, landmark detection, and similar body part recognition and tracking functionality. SCIPY, a Python-based peak finding algorithm can be used for two dimensional excursion calculation from the landmark motion, to determine the peak positions of fingers or other body parts during a twitching motion. It will be appreciated that although calculation of the TOF ratio is discussed herein with respect to measurement of twitches in the first and fifth finger, other body parts can be used to assess TOF ratio, such as but not limited to around the eye and ankle.

The computer vison model 42 may include a data entity that describes parameters, and/or defined operations of a rules-based, machine learning model, and/or artificial intelligence model (e.g., model including at least one of one or more rule-based layers, one or more layers that depend on trained parameters, coefficients, and/or the like) configured to identify body parts in image data and track the movement of the body parts. In this regard, in some embodiments, the computer vision model 42 may be configured to utilize one or more of any type of machine learning, rules-based, and/or artificial intelligence techniques including one or more of computer vision techniques, supervised learning (e.g., using user feedback), unsupervised learning, semi-supervised learning, reinforcement learning, computer vision techniques, sequence modeling techniques, neural network techniques, and/or other artificial intelligence techniques. For example, the computer vision model 42 may be configured to employ computer vision techniques to analyze one or more images or videos to identify certain body parts, such as the first finger (thumb) and the fifth finger (pinky finger) of a hand, and/or to track the movement thereof.

Customized machine learning models require extensive training that relies on the generation of large volumes of training data. These data are expensive and time consuming to generate or acquire. Proper training involves significant attention paid to the generalizability and diversity of the training data to ensure clinical relevance. Validating machine learning models in the clinical setting is time consuming and expensive, requiring extensive design and testing and can be a burden to overall development.

The computer vision model 42 according to certain example embodiments may therefore be trained with simulated image data, as discussed in further detail herein. High Fidelity simulation of the anesthesia environment and of human anatomy and motion is now achievable with advances in graphics hardware and video game design engines. Video of these environments can be simulated and generated for analysis and a machine learning workflow. For example, the Unity® game engine can be used to generate a high-fidelity patient model in a simulated domain and to train and evaluate the computer vision model 42. These simulations enable the production of both testing and validation data sets with fewer resources than their real world counterparts, providing an efficient implementation of the computer vision model 42. Large batches of training data can be systematically generated and their ground truth values, or labels, recorded. The training data are processed by the computer vision model 42 to train the model to recognize specific types of body parts, such as the first and fifth fingers, in a three-dimensional space, and track their movements.

The neuromuscular blockade assessment apparatus 40 may be implemented wholly, or partially on the user device 30, or may be implemented as a server configured to communicate with the user device 30. In this regard, it will be appreciated that certain functionality can be implemented at the user device 30 to provide edge processing advantages, such as not limited to faster response times, continued functionality even during poor network connectivity, and the like. However, certain functionality may be implemented at a remote server or other computing device to provide advantages of server-based processing, including increased computational power, control over updates, and less mobile device strain.

According to certain embodiments, the neuromuscular blockade assessment apparatus 40 may be integrated with an electronic health record (EHR). The information collected or generated by the neuromuscular blockade assessment apparatus 40 can be imported into a patient chart, and utilized in patient health summaries, test results, and the like. According to certain example embodiments in which the neuromuscular blockade assessment apparatus 40 or portion thereof is implemented on the user device 30, such as a smart phone, the neuromuscular blockade assessment apparatus 40 may be integrated with a mobile application designed for practitioners and other clinical users to access patient data, review charts, and manage tasks.

The neuromuscular blockade assessment apparatus 40, including the computer vision model 42, is utilized in the medical field to receive image data from the user device 30 and/or camera 32, track movement of body parts, calculate a twitch count or a quantitative TOF ratio, and generate a quantitative neuromuscular blockade assessment. According to certain example embodiments, the neuromuscular blockade assessment apparatus 40 may be used to control an external device 50, to calculate a dosage of a reversal agent, based on the neuromuscular blockade assessment. In this regard, the neuromuscular blockade assessment can include a calculated dosage of the reversal agent or identify that acceptable recovery has occurred and no reversal agent is required. The external device 50 may therefore include an infusion pump, a syringe, a controlling device of the infusion pump and/or syringe, and/or any combination thereof, configured to administer, or control administration of a muscle relaxant or reversal agent.

The system of FIG. 1 described above is provided merely as an example implementation and it will be appreciated that the example embodiments provided herein may be implemented as or employed by any number of system architectures. For example, the system of FIG. 1 can be distributed amongst different computing devices in a variety of ways.

Referring now to FIG. 2, apparatus 200 is a computing device(s) configured for implementing any of the user device 30, camera 32, neuromuscular blockade assessment apparatus 40, computer vision model 42, and/or external device 50. Apparatus 200 may at least partially or wholly embody any of the user device 30, camera 32, neuromuscular blockade assessment apparatus 40, computer vision model 42, and/or external device 50.

Apparatus 200 is an example computing entity in accordance with certain example embodiments. In general, the terms apparatus, computing entity, computer, entity, device, system, and/or similar words used herein interchangeably may refer to, for example, one or more computers, computing entities, desktops, mobile phones, tablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. Such functions, steps/operations, and/or processes may include, for example, transmitting, receiving, operating on, processing, displaying, storing, determining, creating/generating, monitoring, evaluating, comparing, and/or similar terms used herein interchangeably. In one embodiment, these functions, steps/operations, and/or processes can be performed on data, content, information, and/or similar terms used herein interchangeably.

Apparatus 200 may include or otherwise be in communication with a processor 210, user interface 212, communication interface 214, and memory 216. As described above, apparatus 200 may be implemented as a distributed system for performing the operations described herein. As such, any of the components such as the processor 210, user interface 212, communication interface 214, and memory 216, or portion(s) thereof, may be distributed across multiple computing devices and may be collectively configured to operate as apparatus 200. As such, the various operations described herein may indeed be performed by different computing devices.

The processor 210 may, for example, be embodied as various means including one or more microprocessors with accompanying digital signal processor(s), one or more processor(s) without an accompanying digital signal processor, one or more coprocessors, one or more complex programmable logic devices (CPLDs), one or more multi-core processors, one or more controllers, processing circuitry, one or more computers, various other processing elements including integrated circuits such as, for example, an ASIC (application specific integrated circuit) or FPGA (field programmable gate array), or some combination thereof. Accordingly, although illustrated in FIG. 2 as a single processor, in some embodiments, processor 210 comprises a plurality of processors. The plurality of processors may be embodied on a single computing device, such as server 40, or may be distributed across a plurality of computing devices collectively configured to function as the processor 210. The plurality of processors may be in operative communication with each other and may be collectively configured to perform one or more functionalities as described herein. In an example embodiment, processor 210 is configured to execute instructions stored in memory 216 or otherwise accessible to processor 210. These instructions, when executed by processor 210, may cause the apparatus 200 to perform one or more of the functionalities as described herein.

Whether configured by hardware, firmware/software methods, or by a combination thereof, processor 210 may comprise an entity capable of performing operations according to the example embodiments described herein. Thus, for example, when processor 210 is embodied as an ASIC, FPGA or the like, processor 210 may comprise specifically configured hardware for conducting one or more operations described herein. Alternatively, as another example, when processor 210 is embodied as an executor of instructions, such as may be stored in memory 216, the instructions may specifically configure processor 210 to perform one or more algorithms and operations described herein.

As will therefore be understood, the processor 210 may be configured for a particular use or configured to execute instructions stored in one or more memory elements including, for example, one or more volatile memories and/or non-volatile memories such as memory 216. As such, whether configured by hardware or computer program products, or by a combination thereof, the processor 210 may be capable of performing steps or operations according to embodiments of the present disclosure when configured accordingly. The processor 210, for example in combination with the one or more volatile memories and/or or non-volatile memories, may be capable of implementing one or more computer-implemented methods described herein, such as one performed responsive to executing computer program code.

For example, the processor 210 may be configured to generate a body part movement data object based on image data (e.g., image data and/or video data) received from the one or more cameras, describing the movement of a body part. Additionally, or alternatively, the processor 210 is be configured to receive a body part movement data object representative of a position of one or more body parts in a three-dimensional space, wherein the body part movement data object is generated based on data received from one or more cameras. The processor 210 is configured to apply a computer vision model to the body part movement data object to calculate a TOF ratio. Based upon the calculated TOF ratio, the processor 210 generates a neuromuscular blockade assessment.

According to certain embodiments, the processor 210 is further configured to generate and provide an alert based on the neuromuscular blockade assessment. The alert may be provided via communication interface 214, and provided as output to a user via user interface 212. The processor 210 may be configured to train the computer vision model using one or more simulated body parts and generated video frames of the one or more simulated body parts.

Memory 216 may comprise, for example, volatile memory, non-volatile memory, or some combination thereof. Although illustrated in FIG. 2 as a single memory, memory 216 may comprise a plurality of memory components. The plurality of memory components may be embodied on a single computing device or distributed across a plurality of computing devices. Memory 216 may include a database, for example, and/or any memory components of user device 30, camera 32, neuromuscular blockade assessment apparatus 40, computer vision model 42, and/or external device 50. In various embodiments, memory 216 may comprise at least a non-transitory medium such as but limited to a hard disk, random access memory, cache memory, flash memory, a compact disc read only memory (CD-ROM), digital versatile disc read only memory (DVD-ROM), an optical disc, circuitry configured to store information, or some combination thereof. Memory 216 may be configured to store information, data (including image repositories, database tables, etc.), applications, computer program product, instructions, or the like for enabling apparatus 200 to carry out various functions in accordance with example embodiments described herein. For example, in at least some embodiments, memory 216 is configured to buffer input data for processing by processor 210. Additionally, or alternatively, in at least some embodiments, memory 216 is configured to store program instructions for execution by processor 210. Memory 216 may store information in the form of static and/or dynamic information. This stored information may be stored and/or used by apparatus 200 during the course of performing its functionalities.

A computer program product stored on memory 216 may include one or more software components including, for example, software objects, methods, data structures, or the like. A software component may be coded in any of a variety of programming languages. An illustrative programming language may be a lower-level programming language such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as in a particular directory, folder, or library. Software components may be static (e.g., pre-established or fixed) or dynamic (e.g., created or modified at the time of execution).

A computer program product may include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

According to certain embodiments, memory 216 includes a non-volatile computer-readable storage medium, such as a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

According to certain embodiments, memory 216 includes a volatile computer-readable storage medium such as random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

Communication interface 214 may be embodied as any device or means embodied in circuitry, hardware, a computer program product comprising computer readable program instructions stored on a computer readable medium (e.g., memory 216) and executed by a processing device (e.g., processor 210), or a combination thereof that is configured to receive and/or transmit data from/to another device and/or network, such as, for example, a second apparatus 200 and/or the like. In some embodiments, communication interface 214 (like other components discussed herein) can be at least partially embodied as or otherwise controlled by processor 210. In this regard, communication interface 214 may be in communication with processor 210, such as via a bus. Communication interface 214 may include, for example, an antenna, a transmitter, a receiver, a transceiver, network interface card and/or supporting hardware and/or firmware/software for enabling communications with another local or remote computing device and/or servers. Communication interface 214 may include a network (e.g., network 10), such as any wired or wireless communication network including a local area network (LAN), personal area network (PAN), wide area network (WAN), the Internet, an intranet, or the like, as well as any attendant hardware, software and/or firmware required to implement said networks (e.g. network routers and network switches).

Communication interface 214 may be configured for communicating with various computing entities, such as by communicating data, content, information, and/or the like that can be transmitted, received, operated on, processed, displayed, stored, and/or the like. Such communication data may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the communication interface 214 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Communication interface 214 may be configured to receive and/or transmit any data that may be provided by computing devices, for example, using any protocol that may be used for communications between computing devices. Communication interface 214 may be further configured, for example, to write data to memory 216. Communication interface 214 may additionally or alternatively be in communication with the memory 216, user interface 212 and/or any other component of apparatus 200, such as via a bus.

User interface 212 may be in communication with processor 210 to receive an indication of a user input and/or to provide an audible, visual, mechanical, or other output to a user. As such, user interface 212 may include, for example, a keyboard, a mouse, a user device, a computer, a display, a speaker, a microphone, and/or other input/output mechanisms. In embodiments in which apparatus 200 is embodied as a distributed system, user interface 212 may be implemented on a user device, such as user device 30, that may be separate from a server or other computing device configured to perform at least some of the operations described herein. For example, at least some aspects of user interface 212 may be embodied on an apparatus used by a user that is in communication with apparatus 200. For example, the user interface 212 may be implemented at least partially on a user device, such as user device 30, and may be configured for viewing information or notifications pertaining to a neuromuscular blockade assessment.

The user interface 212 may include a browser or graphical user interface presented by a mobile application. The user interface 212 can comprise any of a number of input devices or interfaces allowing the apparatus 200 to receive data including, as examples, a keypad (hard or soft), a touch display, voice/speech interfaces, motion interfaces, and/or any other input device. In embodiments including a keypad, the keypad can include (or cause display of) the conventional numeric (0-9) and related keys (#, *, and/or the like), and other keys used for operating the apparatus 200 and may include a full set of alphabetic keys or set of keys that may be activated to provide a full set of alphanumeric keys.

The user interface 212 may be in communication with memory 216, communication interface 214, and/or any other component(s), such as via a bus. One or more than one user interfaces 212 can be included in apparatus 200.

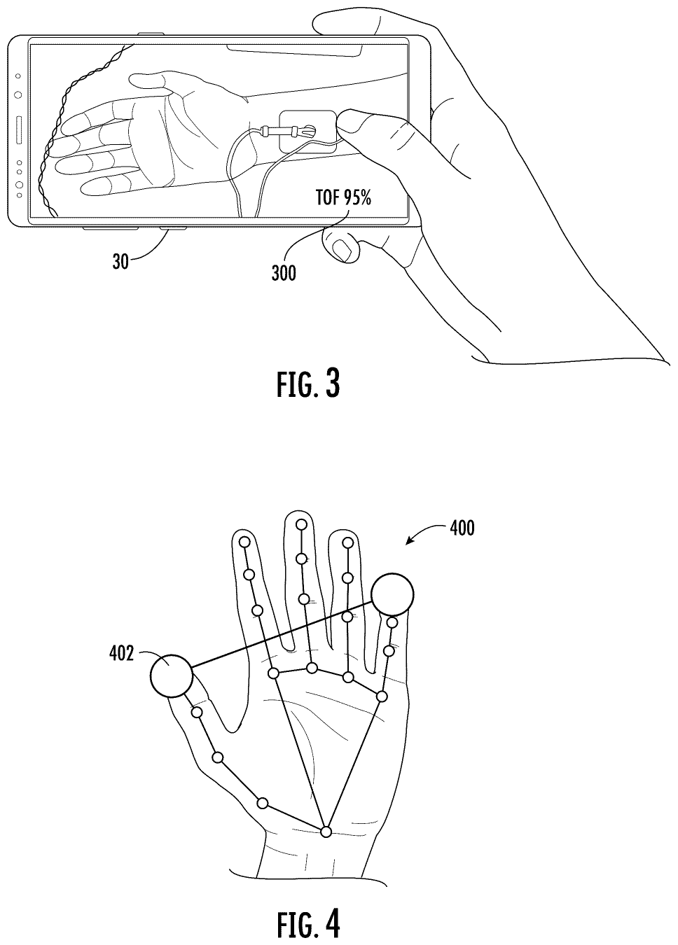

FIG. 3 is an example illustration of a user device 30 in use by a user, according to certain example embodiments. A medical practitioner or other user holds the user device 30 such that a camera 32 of the user device 30 captures image data of a patient's body part, such as their hand. As an electrical stimulus is applied to the patient, the camera 32 captures the twitching movement of the patient's observed body part that is responding to the electrical stimulus. With the computer vision approach any electrical stimulator can be used to elicit the train of four or twitch count while the computer vision function is activated on the user device 30. According to certain example embodiments, a user may initiate the process by a button, click, or icon tap to start the computer vision to look for a stimulated site and then a twitch count or TOF ratio when there are four twitches.

The neuromuscular blockade assessment apparatus 40, with the computer vision model 42, recognizes the stimulated site, such as a hand, and monitors the frames of the image data, and detects the various points in a patient's e.g. hand, as illustrated in FIG. 4. The motion and peaks of the patient's fifth finger 400 and first finger 402 are tracked. The neuromuscular blockade assessment apparatus 40, can then monitor the twitch count and/or train of four ratio and generate the quantitative neuromuscular blockade assessment. At a particular time during monitoring, the twitch count can be 0, 1, 2, 3, 4, etc. The more profound the neuromuscular blockade, the more likely the count is zero and then with less deep block the count progressively becomes 1, then 2, then 3 and then 4 at which point example embodiments can calculate the TOF ratio and generate a neuromuscular blockade assessment

According to certain embodiments, an initial cycle of the computer vision activation of a TOF assessment can be to establish a baseline TOF ratio or an assessment of TOF ratio without a referencing baseline. The assessment can then be reported and/or displayed in the form of a number, such as the TOF ratio or twitch count displayed to the user at 300. According to certain embodiments, a bar graph illustrating the TOF ratio or twitch count can be displayed. If captured, the display can also include the baseline TOF ratio, such as pre-muscle relaxant administration. Various data points, such as baseline TOF ratio and most recent TOF ratio can be displayed in different shades, or adjacent to one another. Subsequent TOF ratios can be overlaid or displayed adjacently to prior TOF ratios to allow for visual comparison. The reference baseline can be adjacent or in a different shade with the current ratio in % or fraction displayed as an absolute numerical value or it can be normalized as a % or fraction of the baseline value (if a baseline was obtained). Note that the % or fraction may be greater than 100% when percentage is used, or greater than 1 if fractions or ratios are used.

In a more passive arrangement, a camera 32, such as a peripheral camera, can be set up for monitoring, such as on a continuous basis, or in synchronization with an electrical stimulation device. A notification or alert can be produced at a user device 30, which can be in proximity to the patient, and/or remotely configured.

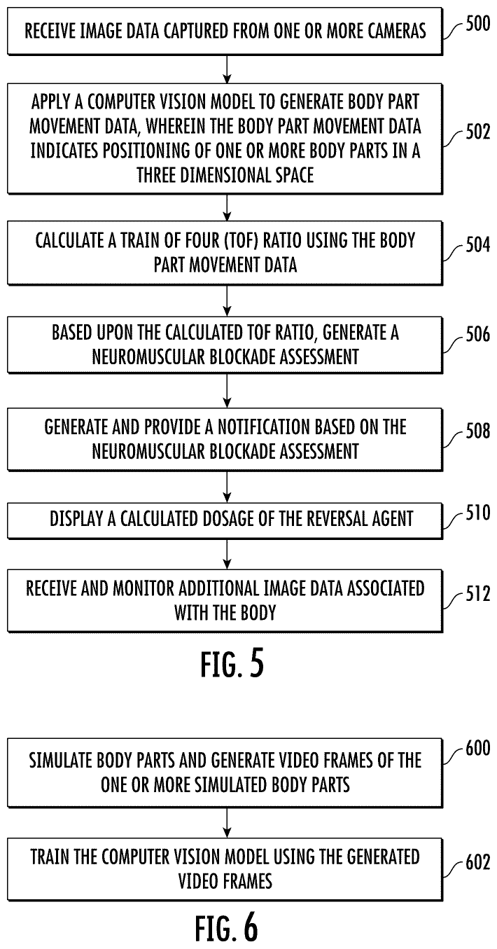

FIG. 5 is a flowchart of operations performed by apparatus 200, such as neuromuscular blockade assessment apparatus 40, according to some example embodiments. As shown by operation 500, apparatus 200, such as with processor 210, user interface 212, communication interface 214, memory 216, and/or the like, receives image data captured from one or more cameras. As shown in FIG. 3, one or more cameras 32, which may be comprised in a user device 30, are positioned facing toward a patient and/or a body part and directed to capture video. The image data are transmitted to the neuromuscular blockade assessment apparatus 40, such as via the user device 30. The neuromuscular blockade assessment apparatus 40 therefore receives the image data captured from the one or more cameras. According to example embodiments in which the neuromuscular blockade assessment apparatus 40 is embodied by the user device 30, the image data may not necessarily be transmitted externally from the user device 30, but are received by the neuromuscular blockade assessment apparatus 40 for further processing as described herein.

At operation 502, apparatus 200, such as with processor 210, communication interface 214, memory 216 and/or the like, applies a computer vision model 42 to generate body part movement data, wherein the body part movement data indicate positioning of one or more body parts in a three-dimensional space. Generating body part movement data may include identifying key points on the body, such as the first and fifth fingers of a patient's hand, the eye, the foot.

Generating the body part movement data may further include tracking movement of body parts, such as the first and fifth fingers, and identifying peak positions. For example, peak positions may include positions of the first and fifth fingers when the first and fifth fingers are the furthest apart and closest together. The first and fifth finger movement, or twitching, is used as an example, and it will be appreciated that the body part movement data may apply to other body parts, such as eyelids/periorbital muscles, and/or the like. The body part movement data may therefore include positions of body parts at various points in time and may further include indicators of the peak and at rest positions.

In a medical field environment, various factors can impact consistency and clarity of image frames of a patient's moving body parts, such as fingers. The patient's hand may be resting on a bed at an angle, making it difficult for the camera to capture the first and fifth fingers. Blankets, bedrails, and other objects may block parts of the patient's hand. Variation in skin color, background colors, viewpoint angles, and the like can make it further challenging for the computer vision model 42 to distinguish the fingers in a three-dimensional space and identify peaks in movements as the fingers twitch. According to certain example embodiments, a fiducial marker can be placed on body parts, such as e.g. the first or alternatively first and fifth fingers, to improve or optimize the recognition and tracking capabilities of the computer vision model 42. The fiducial marker can be an inexpensive, disposable or one-time use marker, that along with the computer-based implementation of the neuromuscular blockade assessment apparatus 40, provides an efficient, inexpensive, and accurate solution to TOF ratio and twitch count monitoring.

At operation 504, apparatus 200, such as with processor 210, communication interface 214, memory 216 and/or the like, calculates a twitch count and/or train of four (TOF) ratio using the body part movement data. In this regard, the neuromuscular blockade assessment apparatus 40 identities first, second, third, and then fourth sets of body part movement data in sets of body part movement data associated with sequential peak positions. If there are four movements elicited by a twitch stimulus then a train of four ratio is quantified. The neuromuscular blockade assessment apparatus 40 can therefore calculate a quantitative TOF ratio as the ratio in amplitude of the fourth twitch compared to the first of a series of four twitches.

Utilizing the neuromuscular blockade assessment apparatus 40 and the computer vision model 42 to calculate a quantitative TOF ratio provides improvements over alternative clinical (visual, tactile) methods for estimating the TOF ratio. Example embodiments provide a quantitative solution to TOF ratio estimation. By using a computer vison model 42, example embodiments reduce reliance on expensive, high-maintenance sensors such as single-use or disposable electrodes. Example embodiments can be implemented on smart phones devices which can minimize costs and improve technology pertaining to medical data reporting and monitoring. Even if a disposable fiducial marker is used, the cost is substantially lower than that of disposable sensors, and is not reliant on any special configuration and/or calibration of sensors or user training.

At operation 506, apparatus 200, such as with processor 210, communication interface 214, memory 216 and/or the like, generates a neuromuscular blockade assessment based upon the twitch count or if there are four twitches then the calculated TOF ratio. The neuromuscular blockade assessment can be generated in a variety of ways and can include various information relating to neuromuscular blockade. According to certain embodiments, the neuromuscular blockade assessment can be a quantitative assessment, such as the TOF ratio, a quantitative assessment of acceptable recovery from neuromuscular blockade, recovery, calculated dosage of a neuromuscular blockade reversal agent, or the like.

According to certain example embodiments, an indicator indicating whether a patient is acceptably recovered can be included in the neuromuscular blockade assessment. For example, if a TOF ratio of 90% or greater of the baseline TOF ratio is reached, the neuromuscular blockade assessment is generated to include an indicator of recovery, meaning a patient has recovered to an acceptable level, can be safely extubated from a neuromuscular blockade recovery standpoint, and/or the like. 90% is used as a threshold acceptable recovery ratio and it will be appreciated that other thresholds may be used as a basis for generating the neuromuscular blockade assessment.

According to certain embodiments, a TOF ratio <90% of baseline is indicative of residual neuromuscular blockade, such that a reversal agent is recommended if for example the patient is to be extubated. Residual neuromuscular blockade is considered to occur when the TOF ratio is below 90% of the baseline TOF ratio, e.g. 82%. In this regard, according to certain example embodiments, a predefined algorithm can be used to calculate the dosage of a reversal agent, based on the quantitative TOF ratio. A dosage can be recalculated as the TOF ratio continues to be monitored and the patient's reaction to the reversal agent is accounted for based on the TOF ratios, and as the patient's progress is estimated based on the quantitative neuromuscular blockade assessment generated by the neuromuscular blockade assessment apparatus 40.

According to certain example embodiments, a baseline TOF ratio for a patient can be used to monitor patient progress. In this regard, the operations of FIG. 5 may be repeated such that a patient's twitch count or when there is sufficient recovery for there to be 4 twitches then the TOF ratio can be monitored over a period of time. An initial TOF ratio prior to any neuromuscular blocking drug administration may be considered a baseline TOF ratio. In this regard, a neuromuscular blockade assessment regarding recovery, residual blockade, and/or the like can be personalized based on a patient's progress and baseline or prior TOF ratios. According to an example embodiment, a neuromuscular blockade assessment may indicate expected or healthy progress toward recovery, even if the 90% or other threshold TOF ratio has not yet been achieved, such that the neuromuscular blockade assessment does not provide reason for action even though the threshold has not yet been achieved.

According to another example, if a patient's progress suddenly drops from a trending recovery, the neuromuscular blockade assessment may include an indicator of the change in trend. Numerous variations of assessments can be contemplated using the monitored TOF ratio for a patient. Using the neuromuscular blockade assessment apparatus 40 according to example embodiments provides for improved efficiencies, in comparison to other monitoring techniques that require several steps, such as estimating the TOF ratio, and comparing it to prior TOF ratios for the patient. Monitoring the TOF ratio on a continual basis is impractical to perform by clinical methods or in the mind, and example embodiments provided herein provide an efficient and accurate quantitative solution to monitoring neuromuscular blockade.

At operation 508, apparatus 200, such as with processor 210, user interface 212, communication interface 214, memory 216 and/or the like, for generating and providing a notification based on the neuromuscular blockade assessment. The neuromuscular blockade assessment apparatus 40 generates a notification based on the neuromuscular blockade assessment and the type of information indicated by the neuromuscular blockade assessment.

According to certain embodiments, the notification may be provided with varying levels of prominence or urgency. For example, if a neuromuscular blockade assessment indicates progress toward recovery, the notification can be passive. For example, a most recent patient's TOF ratio, and/or charted progress over a series of TOF ratios can be displayed on a graphical user interface via a user interface 212, on user device 30. For example, the neuromuscular blockade assessment can indicate a last measured TOF ratio and the time thereof.

According to certain example embodiments, if a neuromuscular blockade assessment indicates residual blockade, or other need for intervention, a notification may be more prominent, urgent, or more proactively provided via the user interface 212 in comparison to a notification indicating recovery or progress toward recovery. For example, an audible alarm may be provided via user device 30. Visual indicators may be made displayed with greater prominence, in comparison to a notification indicating recovery or progress toward recovery, such as by varying a color scheme, creating flashing effects, and/or the like.

Notifications provided by the neuromuscular blockade assessment apparatus 40 may be communicated via a mobile application configured for clinical use and access to EHR data. According to certain embodiments, such a notification may be transmitted to additional devices or user devices, based on the urgency of the notification. For example, other user devices 30 configured for such communication, such as via a local area network, may be alerted of the notification. A practitioner or clinician wearing a smart watch, may see a notification pertaining to the neuromuscular blockage assessment. As another example, a centralized workstation in a medical field environment could display statuses of various patients, including notifications pertaining to neuromuscular blockade assessments.

At operation 510, apparatus 200, such as with processor 210, user interface 212, communication interface 214, memory 216 and/or the like, causes display of a calculated dosage of a reversal agent. In this regard, a clinician can view the recommended or calculate dosage and initiate steps to administer the reversal agent. In this regard, example embodiments guide the clinician in preparing and/or administering an appropriate dosage of reversal agent as determined by at least the processor 210.

According to certain embodiments, the processor 210 controls one or more external devices, such as external device 50, to calculate or administer the calculated dosage of the reversal agent. According to certain embodiments, when a neuromuscular blockade assessment includes a calculated dosage of a reversal agent, or the neuromuscular blockade assessment apparatus 40 calculates a dosage based on an assessment, the neuromuscular blockade assessment apparatus 40 sends an instruction to an external device, such as an infusion pump, a syringe, a controlling device of the infusion pump and/or syringe, and/or any combination thereof. Example embodiments may therefore cause calculation or delivery of a reversal or maintenance neuromuscular blocking agent without further involvement from a clinician. Leveraging the neuromuscular blockade assessment apparatus 40 apparatus to provide the instruction to an external device 50, provides for real-time, proactive corrective active responsive to a neuromuscular blockade assessment. The neuromuscular blockade assessment apparatus 40, according to certain embodiments, therefore provides an improvement to medical monitoring and treatment systems by reducing or eliminating time needed for a practitioner or clinician to interpret TOF ratios, calculate dosages of reversal or agonist agents, initiate administration of the agent, and/or maintain a desired depth of neuromuscular blockade (e.g., a TOF ratio less than 30% as an example). The neuromuscular blockade assessment apparatus 40 provides an accurate, quantitative method for delivering the maintenance or reversal agent, reducing or eliminating risk of human error or human delay.

At operation 512, apparatus 200, such as with processor 210, user interface 212, communication interface 214, memory 216 and/or the like, receives and monitors additional image data associated with the body. In this regard, the operations of FIG. 5 can be repeated. The operations can be repeated on a predefined time interval, or based on a severity level of a patient, surgical stage, or any other patient specific or data driven timing. The neuromuscular blockade assessment apparatus 40 can therefore be configured to dynamically coordinate the timing of initiation of electrical stimulus and corresponding monitoring of TOF ratios.

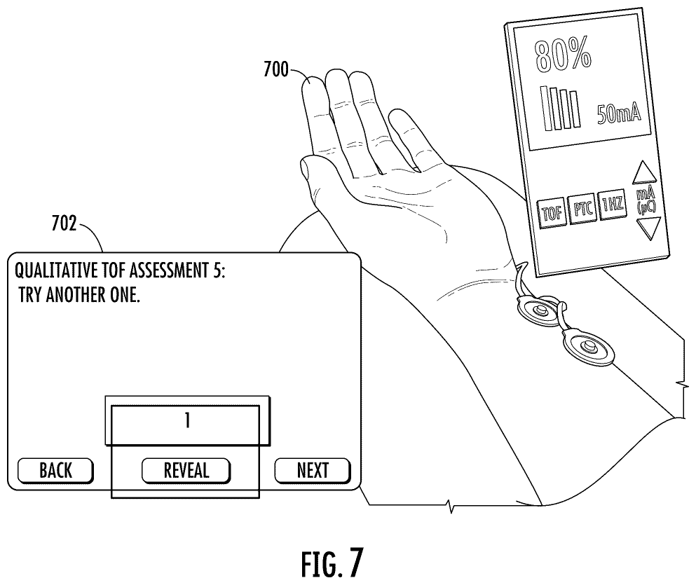

FIG. 6 is a flowchart of operations performed by apparatus 200 according to some example embodiments. The operations of FIG. 6 may be performed by neuromuscular blockade assessment apparatus 40, for example. As shown by operation 600, apparatus 200, such as with processor 210, user interface 212, communication interface 214, memory 216 and/or the like, simulates body parts and movements of the body parts, and generates video frames of the one or more simulated body parts. For example, the Unity® game engine can be used to generate a high-fidelity simulated patient, including a simulated patient hand 700, as shown in FIG. 7. The system can be used to generate video frames of the simulated hand twitching in a manner induced by electrical stimulus. A computer program can be used to systematically or randomly assign input parameters representative of movements of the hand, and the values can be recorded as ground truth labels. According to certain embodiments, parameters can be input to a generated graphical user interface 702. According to certain embodiments, ground truth labels that are TOF ratios and twitch counts can be input to the simulation, and the system generates the simulated movements according to the provided TOF ratios or twitch counts.

As shown by operation 602, apparatus 200, such as with processor 210, communication interface 214, memory 216 and/or the like, trains the computer vision model using the generated video frames and ground truth labels. The computer vision model 42 can therefore learn to identify peak movements of the body part such as the hand, and identify twitching movements used to calculate the TOF ratio, using machine learning algorithms. The simulations enable the generation of both testing and validation data sets with fewer resources than their real world counterparts, providing an efficient implementation of the computer vision model 42 and neuromuscular blockade assessment apparatus 40.

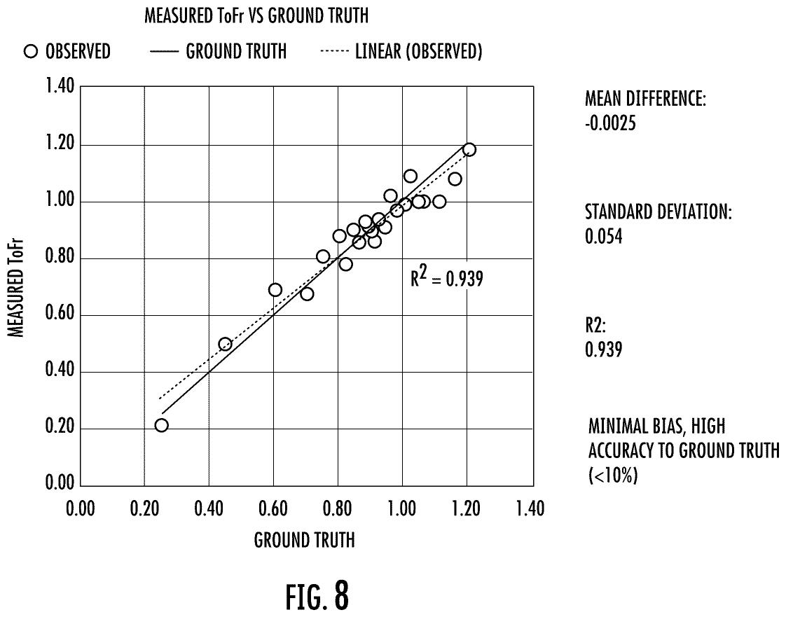

Trials of the neuromuscular blockade assessment apparatus 40 and the corresponding computer vison model 42 consisted of a simulated patient hand made to twitch across a range of TOF ratios. Trials were conducted across TOF ratios of 0.25 to 1.20. Video frames of each trial were analyzed by the OpenCV neural network using the GOOGLE MEDIAPIPE architecture to identify landmarks on the first and fifth fingers and track them moving in three-dimensional space. The resulting coordinates were then analyzed to isolate twitches and determine the distance between the first and fifth finger in each frame. The change in distance was used to calculate the TOF ratio. Results of each trial were regressed against known ground truth values. The mean difference, standard deviation, and coefficient of correlation or determination (R2) were calculated to evaluate accuracy of the neuromuscular blockade assessment apparatus 40 and the corresponding computer vison model 42. FIG. 8 provides a plot of ground truth TOF ratios as generated by a set of example simulations, and the corresponding predicted quantitative TOF ratios determined by the neuromuscular blockade assessment apparatus 40, using the computer vision model 42. FIG. 8 demonstrates a high level of accuracy achieved by the trained computer vision model 42 and corresponding neuromuscular blockade assessment apparatus 40.

By utilizing the computer vision model 42, example embodiments can incorporate more diverse simulated environments on which to train, using more sophisticated and robust motion tracking models, in comparison to using real world image data. Additionally, example embodiments can be adapted to a smartphone platform to provide real-time analysis of twitch count and TOF ratio in the medical field, providing an improved method for monitoring TOF data and providing a proactive corrective action, such as in instances when maintenance or reversal agents are administered in response to the monitoring.

Moreover, the implementation of the neuromuscular blockade assessment apparatus 40 using a user device 30, such as a smart phone, with optional inexpensive and disposable fiducial markers, provides a seamless and easy to setup infrastructure in the medical domain for tracking TOF ratios. In an emergency response scenario, or alternate care site scenario, having the ability to track and monitor TOF ratios with fewer demands on infrastructure enables widespread monitoring with lower demand on infrastructure, equipment, and technology, than alternative methods, such as but not limited to EMG, KMG, PMG and/or AMG implementations that require higher overhead for infrastructure and physical components. Utilizing the computer vision model 42 achieves a high level quantitative analysis that produces more accurate results in a more efficient manner than what can be achieved by a human-observer, while keeping cost and infrastructure demands relatively low in comparison to EMG, KMG, PMG, and/or AMG implementations. Improvements to the overall medical reading and monitoring systems employed in a hospital or other medical domain are therefore provided according to example embodiments.

Example embodiments further provide for easy adaptation in examples in which monitoring of the patient's hand is not feasible. For example, if the patient's arms are tucked, the neuromuscular blockade assessment apparatus 40 can be used to track the patient's reaction to electrical stimulus near the eyes, ankle, or other body part. The clinician can make such an adjustment with little to no invasive impact to the patient, and without requiring additional cumbersome medical equipment or sensors.

FIGS. 5 and 6 illustrate operations of a method, apparatus, and computer program product according to some example embodiments. It will be understood that each operation of the flowchart or diagrams, and combinations of operations in the flowchart or diagrams, may be implemented by various means, such as hardware and/or a computer program product comprising one or more computer-readable mediums having computer readable program instructions stored thereon. For example, one or more of the procedures described herein may be embodied by computer program instructions of a computer program product. In this regard, the computer program product(s) which embody the procedures described herein may comprise one or more memory devices of a computing device (for example, memory 216) storing instructions executable by a processor in the computing device (for example, by processor 210). In some example embodiments, the computer program instructions of the computer program product(s) which embody the procedures described above may be stored by memory devices of a plurality of computing devices. As will be appreciated, any such computer program product may be loaded onto a computer or other programmable apparatus (for example, apparatus 200) to produce a machine, such that the computer program product including the instructions which execute on the computer or other programmable apparatus creates means for implementing the functions specified in the flowchart block(s). Further, the computer program product may comprise one or more computer-readable memories on which the computer program instructions may be stored such that the one or more computer-readable memories can direct a computer or other programmable apparatus to function in a particular manner, such that the computer program product may comprise an article of manufacture which implements the function specified in the flowchart block(s). The computer program instructions of one or more computer program products may also be loaded onto a computer or other programmable apparatus (for example, apparatus 200 and/or other apparatus) to cause a series of operations to be performed on the computer or other programmable apparatus to produce a computer-implemented process such that the instructions which execute on the computer or other programmable apparatus implement the functions specified in the flowchart block(s).

Accordingly, blocks of the flowchart support combinations of means for performing the specified functions and combinations of operations for performing the specified functions. It will also be understood that one or more blocks of the flowchart, and combinations of blocks in the flowchart, can be implemented by special purpose hardware-based computer systems which perform the specified functions, or combinations of special purpose hardware and computer instructions.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A system comprising memory and one or more processors communicatively coupled to the memory, the one or more processors configured to:
receive image data captured from one or more cameras;
apply a computer vision model to generate body part movement data, wherein the body part movement data indicates positioning of one or more body parts in a three-dimensional space;
calculate a twitch count or when there are four twitches a train of four (TOF) ratio using the body part movement data; and
based upon the twitch count or calculated TOF ratio, generate a neuromuscular blockade assessment.

2. The system according to claim 1, wherein the one or more processors are further configured to:
generate and provide a notification based on the neuromuscular blockade assessment.

3. The system according to claim 1, wherein the one or more processors are further configured to:
train the computer vision model using one or more simulated body parts and generated video frames of the one or more simulated body parts.

4. The system according to claim 1, wherein the one or more processors are further configured to:
receive and monitor additional image data, wherein the neuromuscular blockade assessment is generated based on a baseline TOF ratio and the additional image data.

5. The system according to claim 1, wherein the neuromuscular blockade assessment comprises a quantitative assessment.

6. The system according to claim 1, wherein the neuromuscular blockade assessment comprises a calculated dosage of a maintenance or reversal agent.

7. The system according to claim 6, wherein the one or more processors are further configured to:
cause display of the calculated dosage of the maintenance or reversal agent via a user interface.

8. A computer-implemented method comprising:
receiving image data captured from one or more cameras;
applying a computer vision model to generate body part movement data, wherein the body part movement data indicates positioning of one or more body parts in a three-dimensional space;
calculating a twitch count or train of four (TOF) ratio using the body part movement data; and
based upon the twitch count or calculated TOF ratio, generating a neuromuscular blockade assessment.

9. The computer-implemented method according to claim 8, further comprising:
generating and providing a notification based on the neuromuscular blockade assessment.

10. The computer-implemented method according to claim 8, further comprising:
train the computer vision model using one or more simulated body parts and generated video frames of the one or more simulated body parts.

11. The computer-implemented method according to claim 8, further comprising:
receiving and monitoring additional image data, wherein the neuromuscular blockade assessment is generated based on a baseline TOF ratio and the additional image data.

12. The computer-implemented method according to claim 8, wherein the neuromuscular blockade assessment comprises a quantitative assessment.

13. The computer-implemented method according to claim 8, wherein the neuromuscular blockade assessment comprises a calculated dosage of maintenance or reversal agent.

14. The computer-implemented method according to claim 13, further comprising:
causing display of the calculated dosage of the maintenance or reversal agent via a user interface.

15. A computer program product comprising at least one non-transitory computer-readable storage medium having computer-executable program code instructions stored therein, the computer-executable program code instructions comprising program code instructions to:
receive image data captured from one or more cameras;
apply a computer vision model to generate body part movement data, wherein the body part movement data indicates positioning of one or more body parts in a three-dimensional space;
calculate a twitch count or train of four (TOF) ratio using the body part movement data; and
based upon the twitch count or calculated TOF ratio, generate a neuromuscular blockade assessment.

16. The computer program product according to claim 15, wherein the computer-executable program code instructions further comprise program code instructions to:
generate and provide a notification based on the neuromuscular blockade assessment.

17. The computer program product according to claim 15, wherein the computer-executable program code instructions further comprise program code instructions to:
train the computer vision model using one or more simulated body parts and generated video frames of the one or more simulated body parts.

18. The computer program product according to claim 15, wherein the computer-executable program code instructions further comprise program code instructions to:
receive and monitor additional image data, wherein the neuromuscular blockade assessment is generated based on a baseline TOF ratio and the additional image data.

19. The computer program product according to claim 15, wherein the neuromuscular blockade assessment comprises a quantitative assessment.

20. The computer program product according to claim 19, wherein the neuromuscular blockade assessment comprises a calculated dosage of maintenance or reversal agent.
